(19) United States
(12) Patent Application Publication
(10) Pub. No.: US 2009/0105483 A1
(43) Pub. Date: Apr. 23, 2009

(54) PROCESS FOR THE PREPARATION OF PRAMIPEXOLE BASE AND/OR ITS SALTS

(75) Inventors: Roman BALICKI, Warszawa (PL); Michal ODROWAZ-SYPNIEWSKI, Warszawa (PL); Agnieszka CIESIELSKA, Warszawa (PL); Wieslaw SZELEJEWSKI, Warszawa (PL); Joanna ZAGRODZKA, Warszawa (PL); Grazyna CIEPLUCHA, Warszawa (PL)

Correspondence Address: MATTHIAS SCHOLL, 14781 MEMORIAL DRIVE, SUITE 1319, HOUSTON, TX, 77079, US

(73) Assignee: INSTYTUT FARMACEUTYCZNY, Warszawa (PL)

(21) Appl. No.: 12/164,005

(22) Filed: Jun. 28, 2008

Related U.S. Application Data
(63) Continuation-in-part of application No. PCT/PL2006/000093, filed on Dec. 29, 2006.

(30) Foreign Application Priority Data
Dec. 29, 2005 (PL) P.378587

(51) Int. Cl. C07D 277/82

(52) U.S. Cl. 548/164

(57) ABSTRACT

The process for the preparation of pramipexole base and/or its pharmaceutically acceptable salts, especially the hydrochloride salt, in the alkylation reaction of (S)-(−)2,6-diamino-4,5,6,7-tetrahydrobenzothiazole with an alkylating agent, wherein the reaction is carried out in the absence of a base, and in a solvent from which the resulting N-monoalkylated product selectively precipitates out as a salt. After isolation from the reaction mixture, the N-monoalkylated product is converted a) into the free pramipexole base upon treatment with an inorganic base and is then converted into another pharmaceutically acceptable pramipexole salt; or b) directly into another pharmaceutically acceptable pramipexole salt or the hydrate thereof.

### CROSS-REFERENCE TO RELATED APPLICATIONS

This is a continuation-in-part of International Patent Application No. PCT/PL2006/000093, with an international filing date of Dec. 29, 2006, now pending, designating the United States, which is based on Polish Patent Application No. P.378587, filed Dec. 29, 2005. The contents of these specifications including any intervening amendments thereto are incorporated herein by reference.

### BACKGROUND OF THE INVENTION

1. Field of the Invention

This invention relates to a process for the preparation of pramipexole base and/or its pharmaceutically acceptable salts. In particular, the invention relates to the process for preparation of pramipexole base in the reaction of (S)-(−)-2,6-diamino-4,5,6,7-tetrahydrobenzothiazole with an alkylating agent.

2. Description of the Related Art

Pramipexole, i.e., (S)-(−)-2-amino-6-n-propylamino-4,5,6,7-tetrahydrobenzothiazole, is a dopamine receptor agonist with preference for D3 compared to D2 and D4 receptors. Pramipexole dihydrochloride dihydrate is the active ingredient in MIRAPEX® tablets indicated for the treatment of the signs and symptoms of idiopathic Parkinson's disease. Pramipexole is also indicated for the treatment of restless legs syndrome (RLS).

In the European patent application EP 0186087 A1 disclosed are 2,6-diamino-4,5,6,7-tetrahydrobenzothiazole derivatives, and in particular 2-amino-6-n-propylamino-4,5,6,7-tetrahydrobenzothiazole along with its enantiomers and salts of inorganic and organic acids.

In a vast majority of preparative methods described in the literature and patent publications, S-(−)-2,6-diamino-4,5,6,7-tetrahydrobenzothiazole is disclosed as the key intermediate for the synthesis of (S)-(−)-2-amino-6-n-propylamino-4,5,6,7-tetrahydrobenzothiazole (pramipexole base). That intermediate is transformed into pramipexole base by one of two possible two-step processes. A reductive alkylation method is described in European patent application EP 0186087 A1, according to which in the first step, an appropriate Schiff's base is formed using propionyl aldehyde in dimethylformamide, and the Schiff's base is subsequently reduced with sodium borohydride. This claimed procedure is not convenient for a large scale synthesis on account of the very low yield of the expected product.

An alternative two-step procedure comprises acylation of S-(−)-2,6-diamino-4,5,6,7-tetrahydrobenzothiazole with propionyl anhydride followed by a reduction of the 6-acylamino derivative with diborane. Diborane can be either prepared in situ in the reaction of sodium borohydride with boron trifluoride/ethyl ether complex (WO 02/22591), or it can be purchased as a solution in tetrahydrofurane (J. Med. Chem. 30(3), 494-498 (1987)). According to this method, the pramipexole base is obtained in 50% yield. However, this procedure is not without drawbacks. Specifically, the reduction must be carried out with great accuracy (foaming effect is observed) under an atmosphere of an inert gas, the use of a large amount of anhydrous solvent is required, and handling of harmful and toxic reagents is also inevitable. During the reaction, hydrogen is evolved in large quantities, which causes steady danger of explosion in a large scale synthesis.

In the European patent application EP 0186087 A1 disclosed is a different synthetic route to 4,5,6,7-tetrahydrobenzothiazole alkyl derivatives. This route is based on the nucleophilic substitution reaction of alkyl derivatives bearing a halogen or a pseudohalogen substituent (e.g., methoxysulfonylox or p-toluenesulfonyloxy) with a free amine group of 4,5,6,7-tetrahydrobenzothiazole. The alkylation reaction can be performed in wide variety of solvents, such as water, alcohols, cyclic ethers, ketones, acetonitrile, dimethylsulfoxide or neat, optionally in the presence of a base, such as sodium hydroxide, potassium carbonate, sodium hydride, potassium tert-butoxide, or triethylamine.

The procedure for the preparation 2-amino-6-di-n-propylo-4,5,6,7-tetrahydrobenzothiazole is described in the experimental details, wherein the N-dialkylation is carried out in the presence of four molar equivalents of n-propyl bromide and with potassium carbonate as a base. In the example given, time consuming and tedious separation and purification of the product are described. Silica gel purification is necessary in this case due to the fact that under the reaction conditions mono- and polyalkylated derivatives are formed, which are inseparable by other methods.

The synthetic method described in EP 186087 A1 is neither efficient nor selective for the preparation of N-monoalkyl derivatives of 2,6-diamino-4,5,6,7-tetrahydrobenzothiazole using alkyl halogenes and sulfonates.

### BRIEF SUMMARY OF THE INVENTION

In one embodiment, the invention provides a process for the preparation of pramipexole base or its pharmaceutically acceptable salt, or a hydrate thereof, comprising: (a) reacting (S)-(−)-2,6-diamino-4,5,6,7-tetrahydrobenzothiazole with an alkylating agent in a solvent and in the absence of an additional base to yield a salt of an N-monoalkylated product, wherein the salt of the N-monoalkylated product precipitates out from the solvent; (b) isolating the salt of the N-monoalkylated product; and (c) (i) converting the salt of the N-monoalkylated product by treatment with an inorganic base to free pramipexole base and, optionally, converting the free pramipexole base by reacting with an acid or acid equivalent into another pharmaceutically acceptable pramipexole salt; or (ii) converting the salt of the N-monoalkylated product by reacting with an acid or acid equivalent into another pharmaceutically acceptable pramipexole salt, or a hydrate thereof.

In a class of this embodiment, the pharmaceutically acceptable pramipexole salt is pramipexole hydrochloride.

In another class of this embodiment, the solvent is a cyclic or an acyclic tertiary amide, particularly derived from a short chain, C₁-C₃ carboxylic acid, and is more particularly N,N-dimethylformamide, N,N-diethylformamide, N,N-dimethylacetamide, N,N-diethylacetamide, N-methylpirolidone, or their mixture.

In another class of this embodiment, the alkylating agent is represented by the formula 2;

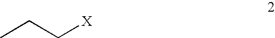

X represents a halogen, or —OSO₂R; and R represents C₁-C₃ alkyl or aryl; the aryl being optionally substituted with a group selected from CH₃, CF₃, F, Cl, Br, I, NO₂, OCH₃, OC₂H₅ or phenyl; particularly, R is α-naphthyl, β-naphthyl, phenyl, or tolyl.

In another class of this embodiment, in step a) (S)-(−)-2,6-diamino-4,5,6,7-tetrahydrobenzothiazole and alkylating agent are provided in a molar ratio of from about 1:1 to about 1:4

In another class of this embodiment, the alkylating agent is selected from a group consisting of n-propyl p-toluenesulfonate, n-propyl bromide, or n-propyl chloride.

In another class of this embodiment, step a) is carried out in a range of temperatures between 0° C. and 100° C., within in a time period of between 12 and 96 hours.

In another class of this embodiment, step a) is carried out in a single phase solvent system or a biphasic solvent system.

In another class of this embodiment, in step a) the alkylating agent is n-propyl p-toluenesulfonate and the solvent is N,N-dimethylformamide.

In another class of this embodiment, the salt of the N-monoalkylated product is crystalline pramipexole p-toluenesulfonate.

In another class of this embodiment, in step a) the salt of the N-monoalkylated product is pramipexole p-toluenesulfonate; and in step c) converting the pramipexole p-toluenesulfonate by treatment with an inorganic base to free pramipexole base, and converting the free pramipexole base by reacting with an acid or acid equivalent into pramipexole dihydrochloride salt or its hydrate.

In another class of this embodiment, in step a) the salt of the N-monoalkylated product is pramipexole p-toluenesulfonate; and in step c) converting the pramipexole p-toluenesulfonatet by reacting with aqueous hydrochloric acid solution or alcoholic acetyl chloride solution or hydrochloride into pramipexole dihydrochloride or the hydrate thereof.

In a subclass of this class, the process further comprises crystallizing pramipexole dihydrochloride from an alcohol-water mixture.

In another class of this embodiment, in step a) the alkylating agent is n-propyl bromide and the solvent is N-methylpyrrolidone.

In another class of this embodiment, the salt of the N-monoalkylated product is a hydrobromide.

In another class of this embodiment, in step a) the alkylating agent is n-propyl chloride and the solvent is N,N-dimethylformamide.

In another embodiment, the invention provides (S)-(−)-2-amino-6-n-propylamino-4,5,6,7-tetrahydrobenzothiazole p-toluenesulfonate, i.e., pramipexole p-toluenesulfonate.

In another embodiment, the invention provides (S)-(−)-2-amino-6-n-propylamino-4,5,6,7-tetrahydrobenzothiazole hydrobromide, i.e., pramipexole hydrobromide.

In another embodiment, the invention provides (S)-(−)-2-amino-6-n-propylamino-4,5,6,7-tetrahydrobenzothiazole hydrochloride, i.e., pramipexole hydrochloride.

### BRIEF DESCRIPTION OF THE DRAWINGS

FIG. 1 illustrates the chemical structure of (S)-(−)-2,6-diamino-4,5,6,7-tetrahydrobenzothiazole 1;

FIG. 2 illustrates the chemical structure of the alkylating agent 2; and

FIG. 3 illustrates the chemical structure of Pramipexole base.

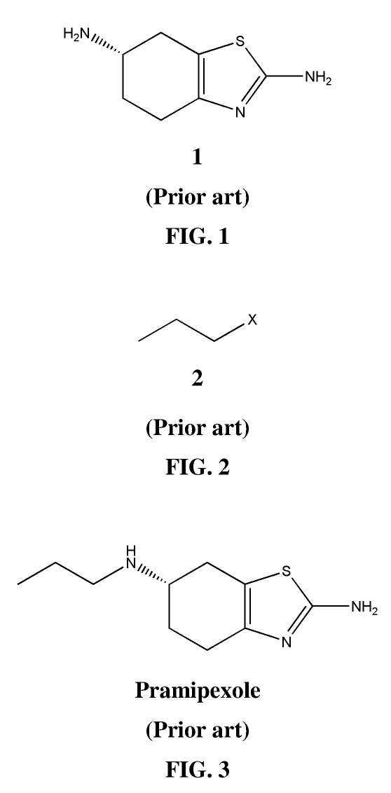

### DETAILED DESCRIPTION OF THE INVENTION

It has now been found that under appropriate conditions, alkylation of (S)-(−)-2,6-diamino-4,5,6,7-tetrahydrobenzothiazole proceeds with high selectivity. This finding not only influences the yield increase but also enables easier separation of the obtained product from the reaction mixture and purification. The background of the invention was the discovery that the alkylation process can be carried out with the absence of a base in a properly-chosen solvent. In that case, the expected product of N-monoalkylation is formed selectively and it precipitates out from the reaction mixture, while by-products, impurities and unreacted substrates remain dissolved in the reaction mixture.

The subject matter of the invention is the process for the preparation of pramipexole base and/or its pharmaceutically accepted salts, especially the hydrochloride salt, in the reaction of (S)-(−)2,6-diamino-4,5,6,7-tetrahydrobenzothiazole with an alkylating agent, wherein the reaction is carried out in the absence of a base, in a solvent from which the resulting N-monoalkylated product selectively precipitates out as a salt.

After isolation from the reaction mixture the N-monoalkylated product: a) is converted into free pramipexole base upon treatment with inorganic base, and then into another pharmaceutically acceptable pramipexole salt, or b) is converted directly into another pharmaceutically acceptable pramipexole salt or the hydrate thereof.

Specifically, it has been found that the N-monoalkylated product selectively precipitates out from solvents, which are the derivatives of cyclic or acyclic tertiary amides of short chain C₁-C₃ carboxylic acids, such as, without limitation, N,N-dimethylformamide, N,N-diethylformamide, N,N-dimethylacetamide, N,N-diethylacetamide, N-methylpirolidone, or their mixtures.

In the methods according to this invention, the appropriate alkylating agent is depicted as having the structure 2 in the drawing, wherein X represents a halogen atom or an —OSO₂—R group; and R represents CIC3 alkyl; or aryl, such as, e.g., α- or β-naphthyl or phenyl, said aryl being optionally substituted with one of the following groups: CH₃, CF₃, F, Cl, Br, I, NO₂, OCH₃, OC₂H₅ or phenyl.

In a preferred embodiment of the invention, the alkylating agent is either substituted or unsubstituted aromatic or aliphatic n-propyl sulfonate, preferably n-propyl p-toluenesulfonate.

Another preferred alkylating agent is n-propyl bromide.

Another alkylating agent is n-propyl chloride.

In the alkylation reaction the molar ratio of (S)-(−)-2,6-diamino-4,5,6,7-tetrahydrobenzothiazole to the alkylating agent is used in the molar range from about 1:1 to about 1:4.

The alkylation process is carried out at the temperatures range of between 0° C. and 100° C., within the time period from 12 to 96 hours. The reaction conditions are adjusted individually, depending on the solvent and alkylating agent used in the reaction following the general rules of organic synthesis.

The reaction can be carried out either in one- or two-phase medium, depending on the amount and type of solvent(s) used.

In a preferred embodiment of the invention, the alkylation reaction is carried out using n-propyl p-toluenesulfonate as the alkylating agent in N,N-dimethylformamide. Under heterogenic reaction conditions, the substrate completely disappears, and (S)-(−)-2-amino-6-n-propylamino-4,5,6,7-tetrahydrobenzothiazole p-toluenesulfonate (pramipexole p-toluenesulfonate) precipitates from the reaction mixture. The expected product forms as a crystalline solid, which can be easily isolated by filtration.

The salt of (S)-(−)-2-amino-6-n-propylamino-4,5,6,7-tetrahydrobenzothiazole with p-toluenesulfonic acid (pramipexole p-toluenesulfonate), which was not described in the literature heretofore, is the next object of this invention.

In another embodiment of the invention, the alkylation reaction is carried out using n-propyl bromide as the alkylating agent in N-methylpyrrolidone. In this particular case, pramipexole forms the salt with hydrobromic acid, which salt precipitates from the reaction mixture as a crystalline solid. The solid is easily separable by filtration.

The salt of (S)-(−)-2-amino-6-n-propylamino-4,5,6,7-tetrahydrobenzothiazole with hydrobromide acid (pramipexole hydrobromide), which was not described in the literature heretofore, is the next object of this invention.

In another embodiment of the invention, the alkylation reaction is carried out using n-propyl chloride as the alkylating agent. As a result, pramipexole hydrochloride is obtained as a crystalline solid.

The salt of (S)-(−)-2-amino-6-n-propylamino-4,5,6,7-tetrahydrobenzothiazole with hydrochloride acid (pramipexole hydrochloride), which was not described in the literature heretofore, is the next object of this invention.

The obtained salt of pramipexole with sulfonic acid or hydrohalogenic acid can be converted into free pramipexole base upon treatment with an aqueous solution of an inorganic base.

Free pramipexole base can further be converted into any pharmaceutically acceptable salt or the hydrate thereof. For instance, to obtain pramipexole dihydrochloride, the free pramipexole base is dissolved in a C₂-C₄ alcohol, e.g., propan-2-ol, and an alcoholic solution of hydrogen chloride is added to the former solution. The forming salt precipitates out from the reaction mixture. The active substance in MIRAPEX®, pramipexole dihydrochloride monohydrate, can be obtained directly, and it meets the requirements for the production of pharmaceuticals. In this case, free pramipexole base is treated with aqueous hydrochloric acid. Pramipexole dihydrochloride monohydrate can be also obtained from the crude anhydrous pramipexole dihydrochloride after crystallization from a water-alcohol solution.

In the preferred embodiment of this invention, the salt of (S)-(−)-2-amino-6-n-propylamino-4,5,6,7-tetrahydrobenzothiazole with p-toluenesulfonic acid is directly converted into pramipexole dihydrochloride during the addition of a solution of acetyl chloride or hydrochloride in an C₂-C₄ aliphatic alcohol or aqueous hydrochloric acid. When using aqueous hydrochloric acid, pramipexole dihydrochloride monohydrate is formed.

The final product can be isolated from the reaction mixture and/or it can be purified by other routinely used laboratory techniques.

The main features of the synthetic procedures described herein are: simplicity, small number of technical operations, and easy isolation of the product, which is free of by-products and impurities. In the process according to the invention, the inconvenient reduction step, used in the prior art methods, as well as the elaborate separation of mono- and dialkylated products are eliminated.

Produced in this way pramipexole free base is of higher purity, above 99.5% (HPLC), and of high optical purity, above 99.90% ee (molar ratio of 99.95:0.05), in comparison with the product purity obtained by other synthetic methods.

### EXAMPLES

This invention is further illustrated by the following non-limiting examples.

### Example 1

### (S)-(−)-2-Amino-6-n-propylamino-4,5,6,7-tetrahydrobenzothiazole, p-toluenesulfonate

The suspension of (S)-(−)-2,6-diamino-4,5,6,7-tetrahydrobenzothiazole 1 (10 g, 0.06 mole) and n-propyl p-toluenesulfonate (38 g, 0.17 mole) in N,N-dimethylformamide (100 mL) was stirred at room temp. for 96 h. Pramipexole p-toluenesulfonate was formed as cream-colored, crystalline solid, which was washed with propan-2-ol. Yield of the product after drying: 14.2 g (64%).

M.p. 253-5° C. (dec.), ¹H NMR (DMSO-d₆) δ: 7,11-7,32 (2d, 4H), 3,50-3,54 (m, 1H), 3,06 (t, 2H), 2,81 (s, 3H), 1,96-3,11 (s, 6H), 2,01 (m, 2H), 0,96 (t, 3H).

### Example 2

### (S)-(−)-2-Amino-6-n-propylamino-4,5,6,7-tetrahydrobenzothiazole, dihydrochloride

a) Direct Method

Pramipexole p-toluenesulfonate (3 g, 0.008 mole) was suspended in propan-2-ol (25 mL). Concentrated hydrochloric acid (1.57 mL) was added portionwise. The mixture was stirred at room temp. for 0.5 h, then for 12 h at 5° C. White, crystalline solid of pramipexole dihydrochloride hydrate was filtered off. Yield of the product 1.48 g (67%).

M.p. 296-8° C.; ¹H NMR (CD₃OD): 3,68 (m, 1H), 3,10 (t, 2H), 2,00-3,28 (m, 6H), 1,95 (m, 2H), 1,06 (t, 3H).

b) Direct Method

To the solution of acetyl chloride (21.4 g, 0.26 mole) in propan-2-ol (20 mL) pramipexole p-toluenesulfonate (3 g, 0.008 mole) was added portionwise. The resulting mixture was stirred for 0.5 h at room temp., then for 12 h at 5° C. White, crystalline solid of pramipexole dihydrochloride was formed, which was filtered off. It was obtained 1.9 g (86%) of the title product, m.p. 297-298° C.

c) Indirect Method—Via Free Pramipexole Base

Pramipexole p-toluenesulfonate (13 g, 0.034 mole) was added to 2% w/w aqueous NaOH solution (100 mL). The resulting mixture was stirred for 3 h at room temp., than pramipexole was extracted with methylene dichloride (3×25 mL). After the removal of the solvent, 7 g (98%) of free pramipexole base was left. It was subsequently dissolved in propan-2-ol (120 mL) and 36% w/w aqueous hydrochloric acid (8 g) was added. Obtained solid was crystallized from propan-2-ol. 8.5 g (90%) of pure pramipexole dihydrochloride hydrate was obtained, m.p. 296-298° C.

### Example 3

### (S)-(−)-2-Amino-6-n-propylamino-4,5,6,7-tetrahydrobenzothiazole, hydrobromide

To the suspension of S-(−)-2,6-diamino-4,5,6,7-tetrahydrobenzothiazole (4.23 g, 25 mmole) in N,N-dimethylacetamide (50 mL) n-propyl bromide was added (12.3 g, 100 mmole) and the resulting mixture was stirred at room temp. for 72 h. A crystalline solid precipitated. It was filtered off and washed in a sep funnel with propan-2-ol (25 mL). After drying 5.82 g of pramipexole hydrobromide was obtained (yield 80%). The product was crystallized from acetone-water (7:3 v/v) solution. M.p. 257-259° C., water contents (Karl-Fischer)-0.2%.

### Example 4

### (S)-(−)-2-Amino-6-n-propylamino-4,5,6,7-tetrahydrobenzothiazole, hydrobromide

To the suspension of S-(−)-2,6-diamino-4,5,6,7-tetrahydrobenzothiazole (4.23 g, 25 mmole) in N-methylpyrrolidone (50 mL) n-propyl bromide (12.3 g, 100 mL) was added and the resulting mixture was stirred at room temp. for 72 h. A crystalline solid precipitated. It was filtered off and washed with propan-2-ol (25 mL). After drying, 6.99 g of pramipexole hydrobromide was obtained (yield 96%).

### Example 5

### (S)-(−)-2-Amino-6-n-propylamino-4,5,6,7-tetrahydrobenzothiazole, hydrochloride

In a thick-wall ampoule, S-(−)-2,6,-diamino-4,5,6,7-tetrahydrobenzothiazole (1.354 g, 8 mmole), N-methylpyrrolidone (16 mL) and n-propyl chloride (2.513 g, 32 mmole) were placed. The contents were stirred at 75° C. for 16 h. After the reaction vessel was cooled to room temp., crystalline solid was filtered off and washed in a sep funnel with propan-2-ol (15 mL). After drying, 0.92 g of pramipexole hydrochloride was obtained (yield 46%). The product was than crystallized from water-acetone solution to give pure pramipexole hydrochloride, m.p. 276-278° C.

### Example 6

### (S)-(−)-2-Amino-6-n-propylamino-4,5,6,7-tetrahydrobenzothiazole, hydrochloride

In a steel autoclave, S-(−)-2,6-diamino-4,5,6,7-tetrahydrobenzothiazole (16.9 g, 0.2 mole), N,N-dimethylformamide (250 mL) and n-propyl chloride (31.4 g, 0.4 mole) were placed. The resulting mixture was stirred at 100° C. for 16 h. After cooling the autoclave to ambient temp., crystalline solid was filtered off and washed in a sep funnel with propan-2-ol. (50 mL). After drying, 10.78 g of pramipexole hydrochloride was obtained as a pale-brown solid (yield 44%). The product was dissolved in water (60 mL), the resulting solution was discolored by addition of charcoal, than condensed and diluted with acetone (150 mL). After the solution was cooled to 5° C., crystalline solid was filtered off. The obtained title product was characterized by m.p. 276-278° C. The water content (Karl-Fischer) was 0.4%.

What is claimed is:

1. A process for the preparation of pramipexole base or its pharmaceutically acceptable salt, or a hydrate thereof, comprising:
(a) reacting (S)-(−)-2,6-diamino-4,5,6,7-tetrahydrobenzothiazole with an alkylating agent in a solvent and in the absence of an additional base to yield a salt of an N-monoalkylated product, wherein said salt of said N-monoalkylated product precipitates out from said solvent;
(b) isolating said salt of said N-monoalkylated product; and
(c) (i) converting said salt of said N-monoalkylated product by treatment with an inorganic base to free pramipexole base and, optionally, converting said free pramipexole base by reacting with an acid or acid equivalent into another pharmaceutically acceptable pramipexole salt; or (ii) converting said salt of said N-monoalkylated product by reacting with an acid or acid equivalent into another pharmaceutically acceptable pramipexole salt, or a hydrate thereof.

2. The process of claim 1, wherein said pharmaceutically acceptable pramipexole salt is pramipexole hydrochloride.

3. The process of claim 1, wherein step a) said solvent is a cyclic tertiary amide or an acyclic tertiary amide.

4. The process of claim 3, wherein said tertiary amide is derived from a short chain, C₁-C₃ carboxylic acid.

5. The process of claim 3, wherein said tertiary amide is selected from N,N-dimethylformamide, N,N-diethylformamide, N,N-dimethylacetamide, N,N-diethylacetamide, N-methylpirolidone, or their mixture.

6. The process of claim 1, wherein
said alkylating agent is represented by the formula 2; 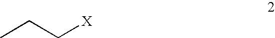
X represents a halogen, or —OSO₂R; and
R represents C₁-C₃ alkyl or aryl; said aryl being optionally substituted with a group selected from CH₃, CF₃, F, Cl, Br, I, NO₂, OCH₃, OC₂H₅ or phenyl.

7. The process of claim 6, wherein R is α-naphthyl, β-naphthyl, or phenyl.

8. The process of claim 1, wherein in step a) (S)-(−)-2,6-diamino-4,5,6,7-tetrahydrobenzothiazole and alkylating agent are provided in a molar ratio of from about 1:1 to about 1:4.

9. The process of claim 1, wherein said alkylating agent is selected from a group consisting of n-propyl p-toluenesulfonate, n-propyl bromide, or n-propyl chloride.

10. The process of claim 1, wherein step a) is carried out in a range of temperatures between 0° C. and 100° C., within in a time period of between 12 and 96 hours.

11. The process of claim 1, wherein step a) is carried out in a single phase solvent system or a biphasic solvent system.

12. The process of claim 1, wherein in step a) said alkylating agent is n-propyl p-toluenesulfonate and said solvent is N,N-dimethylformamide.

13. The process of claim 1, wherein said salt of said N-monoalkylated product is crystalline pramipexole p-toluenesulfonate.

14. The process of claim 1, wherein
in step a) said salt of said N-monoalkylated product is pramipexole p-toluenesulfonate; and
in step c) converting said pramipexole p-toluenesulfonate by treatment with an inorganic base to free pramipexole base, and converting said free pramipexole base by reacting with an acid or acid equivalent into pramipexole dihydrochloride salt or its hydrate.

15. The process of claim 1, wherein
in step a) said salt of said N-monoalkylated product is pramipexole p-toluenesulfonate; and
in step c) converting said pramipexole p-toluenesulfonatet by reacting with aqueous hydrochloric acid solution or alcoholic acetyl chloride solution or hydrochloride into pramipexole dihydrochloride or the hydrate thereof.

16. The process of claim 15, further comprising crystallizing pramipexole dihydrochloride from an alcohol-water mixture.

17. The process of claim 1, wherein in step a) said alkylating agent is n-propyl bromide and said solvent is N-methylpyrrolidone.

18. The process of claim 17, wherein said salt of said N-monoalkylated product is a hydrobromide.

19. The process of claim 1, wherein in step a) said alkylating agent is n-propyl chloride and said solvent is N,N-dimethylformamide.

20. (S)-(−)-2-amino-6-n-propylamino-4,5,6,7-tetrahydrobenzothiazole p-toluenesulfonate.

21. Crystalline (S)-(−)-2-amino-6-n-propylamino-4,5,6,7-tetrahydrobenzothiazole p-toluenesulfonate.
